(19) United States
(12) Patent Application Publication
(10) Pub. No.: US 2018/0303510 A1
(43) Pub. Date: Oct. 25, 2018

(54) Ergonomic Scalpel

(71) Applicant: Elie Levy, Seattle, WA (US)

(72) Inventor: Elie Levy, Seattle, WA (US)

(21) Appl. No.: 15/493,001

(22) Filed: Apr. 20, 2017

(51) Int. Cl. A61B 17/3213

(52) U.S. Cl. CPC A61B 17/3213; A61B 2017/00424

(57) ABSTRACT

An ergonomic scalpel having an elongate body with first end including a blade, and a second end, with the body having a substantially rounded triangular shape in cross section throughout. An elastomeric grip section is proximate to the first end that includes a maximum width of the body, and the body tapers therefrom to the second end. A generally rectangular blade is fastened, either fixedly or removably, to the first end of the body and has a height thereof. The maximum width of the body in the elastomeric grip section is greater than the height of the blade.

### BACKGROUND OF THE INVENTION

### 1. Field of the Invention

The present invention generally relates to hand-held surgical cutting instruments. More particularly, the present invention relates to scalpels having ergonomic form factors and gripping features.

### 2. Description of the Related Art

A scalpel is a small, hand-held, extremely sharp bladed instrument used for surgery, anatomical dissection, and various arts and crafts, often called a hobby knife when used in crafts. Surgical scalpels may have a fixed blade and be single-use and disposable after surgery, or can be reusable through cleaning and sterilization. Disposable fixed-blade scalpels have a metal or polymer handle with a metal or ceramic blade, are used once, then the entire instrument is discarded. Some scalpels can have removable single-use blades that are individually disposable and the body of the scalpel can be sterilized and reused with a new blade.

Scalpel blades are typically made of stainless steel, but can be made from high carbon steel; titanium, ceramic composite, and diamond. Blades can also be made from antimicrobial materials, such as silver or other chemically-infused or coated materials. Some blades can also be coated with a hardener to improve sharpness and edge retention, such as with a zirconium nitride-coating. Blades can also include a coating that improves lubrication and reduces surface tension, such as a polymer coating.

Scalpels typically have an elongated body that is either vertically rectangular or circular in cross-section, such that the scalpel can be held the same way as a writing instrument for precision. The blade is positioned to be at the focal point of one end of the body.

One problem arises in that the body of the scalpel is normally not ergonomic for the hand of the surgeon. The body tends to be very narrow in rectangular cross-section, or in circular cross section, such that the surgeon must maintain a tight pinching motion to cut with the blade, which is painful if one lacks hand-strength or has arthritis or another mild debilitation of the cutting hand.

Another problem arises in that the body of the scalpel tends to be smooth such that particles and liquids cannot penetrate the outer surface of the body, especially in a reusable scalpel. This makes the scalpel slippery to hold, especially when blood or other fluids get on the body and under the grip of the surgeon. Some scalpels include a ridged section or other graspable section on the body near the blade, but the friction created by the section is often inadequate because of the concerns of keeping the body clean.

### SUMMARY OF THE INVENTION

In one embodiment, the present invention is an ergonomic scalpel that has an elongate body having a first end and a second end, with a substantially rounded triangular shape in cross section throughout. The body has a maximum width thereof that tapers from the first end to the second end and an elastomeric grip section is proximate to the first end that includes the maximum width of the body. A blade is fastened to the first end of the body, and is generally rectangular and has a height thereof. The maximum width of the body in the elastomeric grip section is greater than the height of the blade.

The scalpel can be embodied with a fixed blade or a replaceable blade. If replaceable, the first end of the body can include an attachment arm that is configured to releasably attach a blade thereto. Otherwise, the blade can be integrated into the first end of the body and non-releasable. The blade can have a single cutting edge, or double-cutting edges to become a lancet.

The body and elastomeric grip section can be created from a material that withstands autoclaving or other sterilization such that the scalpel is reusable. Further, the body and elastomeric grip section can be made with antimicrobial materials or include an antimicrobial coating. Additionally, the components of the blade, body and elastomeric grip section can be made from recyclable materials such that the scalpel is disposable with minimal environmental impact.

In one embodiment, the taper of the body occurs after the elastomeric grip section and slims inwardly towards the second end. The scalpel can also be embodied such that the body and elastomeric grip section includes a second taper from the maximum width towards the first end of the body.

In use, a surgeon can grip the elastomeric grip section with significant force without utilizing a very-tight pinching motion proximate to the blade, as is common in prior art scalpels. Because the blade has a smaller height than the maximum body width, the surgeon is able to use greater accuracy to guide the blade in the same manner as a precision writing instrument. Due to the lesser tight and consistent force needed to hold the present scalpel, the surgeon runs a lesser risks of cramps or other hand pain during a surgical procedure. Furthermore, the ergonomic aspect facilitates use by surgeons who may be arthritic or have other handicaps.

### BRIEF DESCRIPTION OF THE DRAWINGS

FIG. 1 is a perspective view of one embodiment of the ergonomic scalpel having a replaceable blade.

FIG. 2 is a left-side view of the ergonomic scalpel of FIG. 1.

FIG. 3 is a top view of the ergonomic scalpel of FIG. 1.

FIG. 4 is right side view of the ergonomic scalpel of FIG. 1.

FIG. 5 is a front view of the ergonomic scalpel of FIG. 1.

FIG. 6 is a rear view of the ergonomic scalpel of FIG. 1.

### DETAILED DESCRIPTION

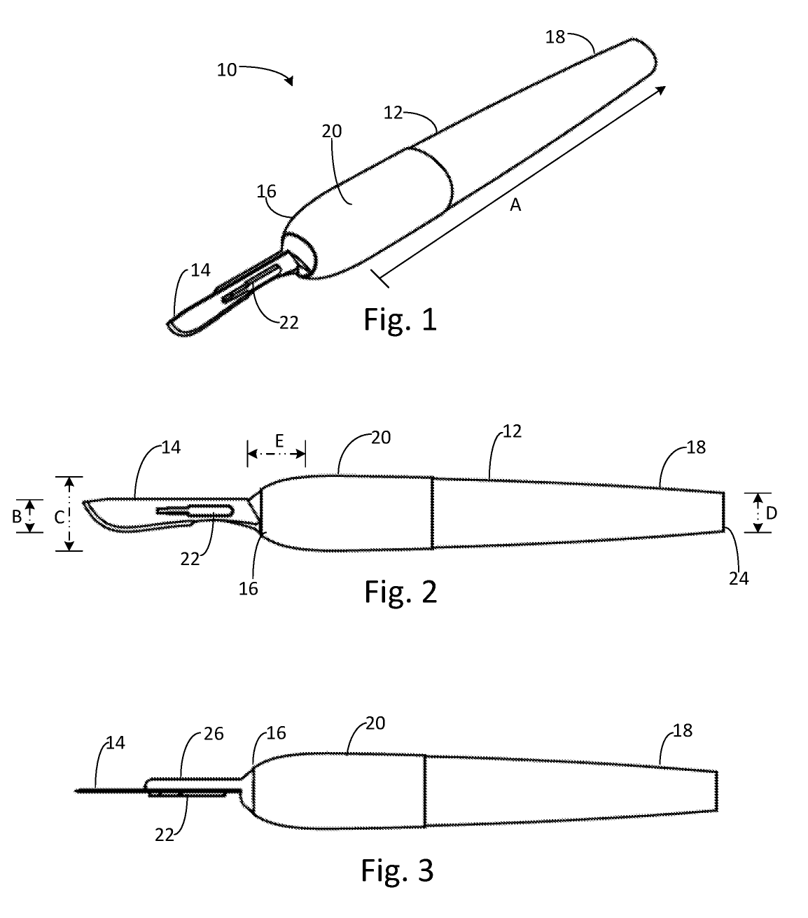

Referring now to the figures in which like numerals represent like elements throughout the several views, FIG. 1 is a perspective view of one embodiment of the ergonomic scalpel 10 that has an elongate body 12 having a first end 16 and a second end 18, with a blade 14 releasable attached to the first end 16. The blade 14 is fitted about an attachment point 22, versions of which are well known in the art. As shown in FIG. 2, the body 12 has a maximum width (Dimension C) thereof that tapers from the first end 16 to the second end 18 and an elastomeric grip section 20 is proximate to the first end 16 that includes the maximum width (Dimension C) of the body 12.

A blade 14 is, here, removably fastened to the first end 16 of the body 12, and is generally rectangular and has a height thereof (Dimension B). The maximum width (Dimension C) of the body 12 in the elastomeric grip section 20 is greater than the height of the blade (Dimension B). The taper occurs in the direction of Arrow A in FIG. 1. The maximum width (Dimension C) is, in this embodiment, about 1.75 cm. The length of the body 12 from blunt end 24 to the tip of the attachment arm 26 (FIG. 3) is about 13.5 cm. The blunt end 24 at the second end 18 is about 9.5 cm in maximum diameter (Dimension D).

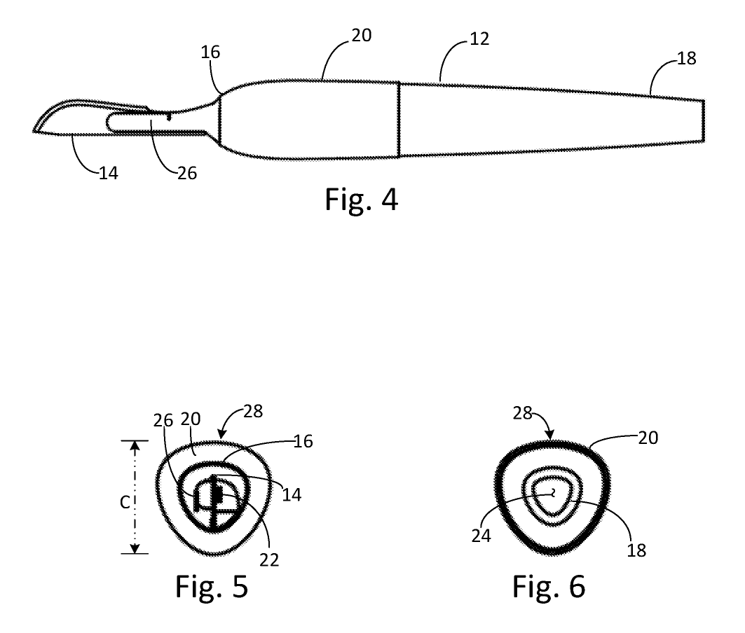

For purposes of further illustration, FIG. 3 is a top view of the ergonomic scalpel 10 of FIG. 1, and FIG. 4 is right side view of the ergonomic scalpel 10 of FIG. 1. FIGS. 3 and 4 illustrate the several positional views of the scalpel 10. FIG. 4 particularly illustrates the attachment arm 26 that holds the removable blade 14.

As shown in FIGS. 5 and 6, the body 12 has a substantially rounded triangular shape in cross section throughout, from the first end 16 to the second end 18. This triangular shape assists in placement of the scalpel 10 between the thumb, index finger, and middle finger, as is the general holding position for those using a scalpel. The index finger can be placed on the top planar surface 28 of the elastomeric grip section 20 to give greater leverage on the blade 14 in cutting.

As shown, the scalpel 10 can be embodied with a fixed blade 14 or a replaceable blade 14. If replaceable, the first end 16 of the body 12 can include an attachment arm 26 that is configured to releasably attach a blade 14 thereto. Otherwise, the blade 14 can be integrated into the first end 16 of the body 16 and non-releasable. The blade 14 can have a single cutting edge, as shown, or can have double-cutting edges to become a lancet.

In the embodiment shown, the taper of the body 12 occurs after the elastomeric grip section 20 and slims inwardly towards the second end 18. The scalpel 10 can also be embodied as shown such that the body 12 and elastomeric grip section 20 includes a second taper (Dimension E in FIG. 2) from the maximum width (Dimension C) towards the first end 16 of the body 12.

The elastomeric grip section 20 can be integrated with the body 12 itself, or formed so as to be flush with the exterior surface of the body 12. Alternately, the elastomeric grip section 20 can be a separate elastomeric material that is adhered upon the body 12 proximate to the first end 16. In such manner, the body 12 can be formed from one solid piece of material and the elastomeric material can then be sprayed, glued, snap-fitted or otherwise fixedly placed on the exterior of the body 12 to form the maximum width (Dimension C) of the elastomeric grip section 20.

The body 12 and elastomeric grip 20 section can be created from a material that withstands autoclaving or other sterilization such that the scalpel 10 is reusable, such as a metal, composite, or rigid polymer for the body 12, and a semi-rigid material for the elastomeric grip section 20 that can withstand the heat and pressure of sterilization. Alternately, the body 12 and elastomeric grip section 20 can be made with antimicrobial materials, such as natural or synthetic polymers, mineral clays, or biologically active metals like silver, copper or zinc. The body 12 and elastomeric grip section 20 could include an antimicrobial coating, such as biocides or silver nanoparticles. Additionally, the components of the blade 14, body 12 and elastomeric grip section 20 can be made from recyclable materials, such as recyclable petroleum or bio-produced polymers and steel, whereby the scalpel 10 is disposable with minimal environmental impact.

Other changes and alterations of the elements of the invention can be made as would be apparent to one of skill in the art without departing from the underlying teachings of the present disclosure.

What is claimed is:

1. An ergonomic scalpel, comprising:
an elongate body having a first end and a second end, the body having a substantially rounded triangular shape in cross section and having a maximum width thereof that tapers from the first end to the second end, the body having an elastomeric grip section proximate to the first end that includes the maximum width of the body;
a blade fastened to the first end of the body, the blade generally rectangular and having a height thereof; and
wherein the maximum width of the body in the elastomeric grip section is greater than the height of the blade.

2. The scalpel of claim 1, wherein the first end of the body further comprises an attachment arm that is configured to releasably attach a blade thereto such that the blade is replaceable.

3. The scalpel of claim 1, wherein the blade is integrated into the first end of the body and non-releasable.

4. The scalpel of claim 1, wherein the body and elastomeric grip section are comprised of a material that withstands autoclaving.

5. The scalpel of claim 1, wherein the body and elastomeric grip section are comprised of an antimicrobial materials.

6. The scalpel of claim 1, wherein the body and elastomeric grip section include an antimicrobial coating.

7. The scalpel of claim 1, wherein the components of the blade, body and elastomeric grip section are comprised of recyclable materials.

8. The scalpel of claim 1, wherein the taper of the body occurs after the elastomeric grip section towards the second end.

9. The scalpel of claim 1, wherein the body and elastomeric grip section includes a second taper from the maximum width towards the first end of the body.

10. The scalpel of claim 1, wherein the blade is a lancet.

11. An ergonomic scalpel, comprising:
an elongate body having a first end and a second end, the body having a substantially rounded triangular shape in cross section and having a maximum width thereof that tapers from the first end to the second end, the body having a gripping means for enhancing the grip of a user, the gripping means proximate to the first end that includes the maximum width of the body;
a cutting means for precision cutting, the cutting means fastened to the first end of the body, and the cutting means having a height thereof; and
wherein the maximum width of the body in the gripping means is greater than the height of the cutting means.

12. The scalpel of claim 11, wherein the first end of the body further comprises an attachment means for releasably attaching a cutting means thereto such that the cutting means is replaceable.

13. The scalpel of claim 11, wherein the cutting means is integrated into the first end of the body and non-releasable.

14. The scalpel of claim 11, wherein the taper of the body occurs after the gripping means towards the second end.

15. The scalpel of claim 11, wherein the body and gripping means includes a second taper from the maximum width towards the first end of the body.

16. An ergonomic scalpel, comprising:
a rigid elongate body having a first end and a second end, the body having a substantially rounded triangular shape in cross section and having a taper from the first end to the second end, the body having an elastomeric grip section adhered upon the body proximate to the first end to thereby creating a maximum width of the body;
a blade fastened to the first end of the body and proximate to the elastomeric grip section, the blade generally rectangular and having a height thereof; and
wherein the maximum width of the elastomeric grip section is greater than the height of the blade.

17. The scalpel of claim 16, wherein the first end of the body further comprises an attachment arm that is configured to releasably attach a blade thereto such that the blade is replaceable.

18. The scalpel of claim 16, wherein the blade is integrated into the first end of the body and non-releasable.

19. The scalpel of claim 16, wherein the taper of the body occurs after the elastomeric grip section toward the second end.

20. The scalpel of claim 16, wherein the body and elastomeric grip section includes a second taper from the maximum width towards the first end of the body.
